(19) United States
(12) Patent Application Publication
(10) Pub. No.: US 2009/0162324 A1
(43) Pub. Date: Jun. 25, 2009

(54) Use of Whole Cell Actinomycetales Bacteria to Treat Stress-Induced Pulmonary Haemorrhage

(75) Inventors: John Lawson Stanford, Kent (GB); Cynthia Ann Stanford, Kent (GB); Graham McIntyre, Kent (GB); Oscar Adelmo Bottasso, Provincia de Santa Fe (AR)

Correspondence Address: STEPTOE & JOHNSON LLP, 1330 CONNECTICUT AVENUE, N.W., WASHINGTON, DC, 20036, US

(73) Assignee: BIOEOS LIMITED, Woodmansterne, Surrey (GB)

(21) Appl. No.: 12/086,420

(22) PCT Filed: Dec. 19, 2006
(86) PCT No.: PCT/GB2006/004787

§ 371 Date: Oct. 28, 2008

(30) Foreign Application Priority Data
Dec. 21, 2005 (GB) 0526032.8

(51) Int. Cl. A61K 35/74

(52) U.S. Cl. 424/93.4

(57) ABSTRACT

Use of whole cells of bacteria from the genera of aerobic organisms in the order of Actinomycetales in the manufacture of a medicament for the treatment or prevention of stress-induced pulmonary haemorrhage (SIPH), preferably exercise induced pulmonary haemorrhage (EIPH), and methods of treating or preventing SIPH, preferably EIPH, in a subject by administering an effective amount of a composition comprising a whole cell of a bacterium from a genera of aerobic organisms in the order of Actinomycetales to said subject. Preferably the aerobic organism is from one or more of the following genera for example: Tsukamurella, Rhodococcus, Gordonia, Nocardia, Dietzia and Mycobacterium.

### FIELD OF INVENTION

The present invention relates to a composition and/or pharmaceutical composition which is effective in the treatment and/or prevention of stress-induced pulmonary haemorrhaging.

### BACKGROUND TO THE INVENTION

Stress-induced pulmonary haemorrhage (SIPH) is a condition that causes bleeding from blood vessels within the lung when the animal is stressed. The term “SIPH” as used herein may encompass the condition HAPE (high altitude pulmonary edema), which is a condition caused by stress-failure of the pulmonary capillaries allowing fluid uptake into the lung (also known as wet-lung). In addition, the term “SIPH” as used herein may also encompasses exercise-induced pulmonary haemorrhage (EIPH), which is a condition that causes bleeding from blood vessels within the lung during strenuous exercise.

HAPE is a potentially fatal condition that typically occurs 2 to 4 days after ascent to altitudes above 3000 m. With usual ascent rates, the incidence is about 1% to 2%, but as many as 10% of people ascending rapidly to 4500 m may develop the condition. HAPE may be preceded by acute mountain sickness, but this is not always the case. The predominant symptom is dyspnea with reduced exercise tolerance. There is often a dry cough at first, but this may progress to a cough that produces frothy, blood stained sputum. Tachypnea and tachycardia are common on examination.

EIPH is known to affect mammals, particularly racing mammals, such as horses, greyhounds, camels and humans. EIPH is known to affect mammals, particularly racing mammals, after intense exercise.

EIPH is most widely described in thoroughbred horses, where it is thought to cause a loss of performance, but has also been observed in standardbred racing (trotting or pacing), polo, show jumping, cross country and barrel racing horses. EIPH is a common condition it is believed afflicting up to 85% of equine athletes.

The symptoms of EIPH vary from minor bleeding detected by observing red blood cells in broncho-alveolar fluid obtained by fibroscopy, to blood appearing in mucus froth around the nostrils at the end of a race. Although in the most severe cases, EIPH manifests as bleeding from the nostrils (epistaxis) but many horses do not show any signs. The use of endoscopy has shown that 40-75% of thoroughbred horses do have blood in their trachea after racing. Diagnosis can also be achieved by tracheal washing and by bronchoalveolar lavage (BAL).

Often SIPH particularly EIPH) worsens with increased age and exercise. EIPH causes financial losses in the racing industry due to reduced performance, loss of training days, medication costs, and banning from racing.

SIPH has also been observed in other organisms such as fish. For example, when a fish is lifted out of water this can cause bleeding from blood vessels within the gills. This phenomenon has been observed in koi carp and can be detrimental to the health of the fish.

The cause of EIPH is still not known. Two current theories are that:
bleeding occurs because horses develop high blood pressures in the pulmonary blood vessels during exercise, leading to stress failure or rupture of these vessels. Scientific evidence that bleeding is due to high blood pressure is poor, and this theory does not explain why haemorrhage tends to be found in the dorso-caudal (upper and rear) part of the lung; and
bleeding is the result of lung damage caused by locomotory impact. The impact of the front legs hitting the ground during racing and jumping is transmitted as a wave to the scapula, which impacts on the chest wall and through the lung tissue and the skeleton. The lung shape results in the wave being amplified and most intense in the dorso-caudal region, where it causes tissue shearing and rupture of blood vessels.

There is no proven effective treatment for EIPH and treatment development has been limited by the uncertainty over the exact cause of EIPH.

The main treatment for EIPH is the use of furosemide (Lasix™) and other diuretics.

Potent diuretics such as furosemide (Lasix™) and ethacrynic acid are administered prior to racing to control EIPH. Lasix™ is legal in the US but is banned in most other countries, and results regarding its effectiveness vary. It is administered intravenously about 4 hours before exercise, and results in increased urine production. The subsequent reduction in blood plasma/blood volume results in a drop in blood pressure, which reduces the severity of bleeding. A horse treated with Lasix™ may also perform better due to the loss of weight by urination, making it a faster horse.

Other treatments that have been considered include the administration of nitric oxide (NO) as a vascular smooth muscle relaxing factor which is thought to mediate endothelial function during exercise. It has been shown to reduce pulmonary artery pressure in horses during strenuous exercise, but in one study the severity of EIPH increased.

EIPH-NOx™ is a device and pharmaceutical treatment combination based on nitric oxide that, once approved, will be the first treatment endorsed by the FDA for EIPH treatment. It is produced by EquATec™, Canada and has been through Phase I feasibility studies.

Other treatments which have been considered are nasal dilators.

External nasal dilator strips, e.g. FLAIR™ nasal strip, have been used to prevent or reduce nasal passage collapse and to decrease nasal resistance (particularly in human athletes). Reduction in EIPH has been demonstrated, but use of strips does not abolish EIPH.

Vasodilators have also been considered. Vasodilators act to enlarge the circumference of the pulmonary vessels, allowing the lung to manage increases in blood flow without stress failure. They include angiotensin converting enzyme (ACE) inhibitors.

Equine concentrated serum—Seramune™—contains equine IgG and other immunoglobulins, and field studies have shown a 62% reduction in EIPH, possibly by an immunotherapy and anti-inflammatory effect.

Herbal and nutritional remedies have also been considered—these include Platinum Performance Equine™ bars, which provide essential nutrients, and PulmonEz™ which stimulates nitric oxide production.

However to date no effective means to treat and/or prevent and/or reduce SIPH, in particular EIPH, has been devised.

### SUMMARY OF THE INVENTION

A seminal finding of the present invention is that stress-induced pulmonary haemorrhaging (SIPH), in particular exercised-induced pulmonary haemorrhaging (EIPH) can be treated and/or prevented and/or reduced by administration of a whole cell of a bacterium from a genus of aerobic organisms in the order Actinomycetales, in particular, by administration of a whole cell of a bacterium from an aerobic Actinomycete.

### DETAILED ASPECTS OF THE INVENTION

In one aspect, the present invention provides the use of a composition comprising whole cells of bacteria from a genus of aerobic organisms in the order Actinomycetales in the manufacture of a medicament for the treatment or prevention of stress-induced pulmonary haemorrhaging.

In a further aspect, the present invention provides a method for treating or preventing stress-induced pulmonary haemorrhaging in a subject comprising administering an effective amount of a composition, preferably a pharmaceutical composition, comprising a whole cell of bacteria from a genus of aerobic organisms in the order Actinomycetales, to a subject.

Suitably, the effective amount of the composition, preferably the pharmaceutical composition, may be administered as a single dose. Alternatively, the effective amount of the composition, preferably the pharmaceutical composition, may be administered in multiple (repeat) doses, for example two or more, three or more, four or more, five or more, six or more, ten or more, twenty or more repeat doses.

In another aspect, the present invention provides a method for protecting, including immunising, a subject from stress-induced pulmonary haemorrhaging comprising administering a pharmaceutical composition comprising whole cells of bacteria from a genus of aerobic organisms in the order Actinomycetales, to the subject.

In a further aspect, the present invention provides a pharmaceutical pack for use in the treatment of stress-induced pulmonary haemorrhage wherein at least one compartment comprises whole cells of bacteria from a genus of aerobic organisms in the order Actinomycetales.

As used herein the terms “stress-induced pulmonary haemorrhage” or “stress-induced pulmonary haemorrhaging” includes “exercise-induced pulmonary haemorrhage” and “exercise-induced pulmonary haemorrhaging”, respectively. Preferably the stress-induced pulmonary haemorrhage is an exercise-induced pulmonary haemorrhage.

In one embodiment the terms “stress-induced pulmonary haemorrhage” or “stress-induced pulmonary haemorrhaging” as used herein also include “high altitude pulmonary edemas”. Preferably the stress-induced pulmonary haemorrhage is a high-altitude pulmonary edema.

The term “whole cells of bacteria from a genus of aerobic organisms in the order of Actinomycetales” as used herein encompasses whole cells of one or more strains of bacteria. Suitably, where the whole cells are from more then one strain of bacteria, said strains may be from one or more genera. And suitably, where said whole cells are from more than one genus, said genera may be from one or more families. Thus, the term encompasses whole cells from a particular strain (for example, whole cells of a particular strain of Tsukamurella inchonensis) and whole cells of bacteria, from a genus of aerobic organisms, from different families (for example, whole cells of strains from Tsukamurella inchonensis and Mycobacterium obuense, which are from the families of Tsukamurellaceae and Mycobacteriaceae, respectively).

Whole cells of many aerobic actinomycete genera can have a corrective influence on immunologically mediated damage.

In one embodiment, suitably the aerobic organism in the order Actinomycetales for use in accordance with the present invention may be from the genus Mycobacterium (such as M. vaccae or M. obuense).

In another embodiment, suitably the aerobic organism(s) in the order Actinomycetales for use in accordance with the present invention may be Nocardioform actinomycetes (such as bacteria mentioned in Group 22 of Bergy's Manual of Determinative Bacteriology, Ninth Edition. Such as, for example, mycolic-acid containing bacteria).

Preferably, the aerobic organism(s) are mycolic acid-containing bacteria (such as bacteria in Group 22 subgroup 1 of Bergy's Manual of Determinative Bacteriology, Ninth Edition. Such as, for example, Tsukamurella, Rhodococcus, Norcardia and Gordonia).

Preferably, the aerobic organism(s) may be from one or more of the following genera: Tsukamurella (such as T. inchonensis and T. paurometabola, preferably from T. inchonensis); Gordonia (such as G. bronchialis, G. amarae, G. sputi and G. terrae, preferably G. bronchialis); Rhodococcus (such as Rhodococcus ruber (previously known as Nocardia rubra), R. rhodnii, R. coprophilus, R. opacus and R. erythopolis, preferably from R. coprophilus) and Norcardia (such as Norcardia asteroides and N. brasiliensis).

In yet another embodiment, suitably the aerobic organism(s) in the order Actinomycetales for use in accordance with the present invention may be from a genus or genera that contain mycolic acid as a component of the cell wall. Examples of such genera include: Tsukamurella, Mycobacterium, Dietzia, Rhodococcus, Norcardia and Gordonia.

Preferably, the aerobic organism(s) in the order Actinomycetales for use in accordance with the present invention is/are from one or more of the following genera: Tsukamurella (such as T. inchonensis and T. paurometabola, preferably from T. inchonensis); Mycobacterium (such as from M. vaccae and M. obuense, preferably from M. obuense); Dietzia (such as Dietzia maris); Rhodococcus (such as from Rhodococcus ruber (previously known as Nocardia rubra), R. rhodnii, R. coprophilus, R. opacus and R. erythopolis, preferably from R. coprophilus); Norcardia (such as from Norcardia asteroides and N. brasiliensis) and Gordonia (such as G. bronchialis, G. amarae, G. sputi and G. terrae, preferably G. bronchialis).

Suitably the aerobic organism(s) may be from the genus Tsukamurella. Preferably, the aerobic organism(s) is/are from T. inchonensis and/or T. paurometabola. Preferably from T. inchonensis.

Suitably the aerobic organism(s) may be from the genus Gordonia. Preferably, the aerobic organism(s) is/are from one or more of the following: G. bronchialis, G. amarae, G. sputi and G. terrae. Preferably from G. bronchialis.

The genus Gordonia used herein may also be referred to as Gordonia. It is intended herein that these terms are interchangeable.

Suitably the aerobic organism(s) may be from the genus Mycobacterium. Preferably, the aerobic organism(s) is/are from M. vaccae and/or M. obuense. Preferably from M. obuense.

A M. obuense strain for use in accordance with the present invention has been deposited by BioEos Limited of 67 Lakers Rise, Woodmansterne, Surrey, SM7 3LA under the Budapest Treaty on the International Recognition of the Deposit of Microorganisms for the purposes of Patent Procedure at the National Collection of Type Cultures (NCTC), Central Public Health Laboratory, 61 Colindale Avenue, London, NW9 5HT) on the 14 Jul. 2005, under Accession Number NCTC 13365.

Suitably the aerobic organism(s) may be from the genus Dietzia. Preferably, the aerobic organism(s) is/are from Dietzia maris.

Suitably the aerobic organism(s) may be selected from the genus Rhodococcus. Suitably, the aerobic organism(s) may be selected from any one or more of the following species: Rhodococcus ruber (previously known as Nocardia rubra), R. rhodnii, R. coprophilus, R. opacus and R. erythopolis. Preferably from R. coprophilus.

Suitably the aerobic organism(s) may be selected from the genus Norcardia. Suitably, the aerobic organism(s) may be selected from any one or more of the following species: Norcardia asteroides and/or N. brasiliensis.

Suitably the bacteria for use in the present invention may be killed prior to use.

In a preferable embodiment a pharmaceutical pack according to the present invention further comprises a label stating that it is suitable for use in the prevention or treatment of stress-induced pulmonary haemorrhage.

Preferably, a course of the composition (preferably the pharmaceutical composition) is administered to an animal known to exhibit pulmonary haemorrhage after stress, prior to exposure to further stress. Initially the course should consist of 2 intradermal injections, preferably given at 2-3 week intervals and preferably being completed 1-3 weeks prior to a fresh application of stress.

In a preferable aspect, subsequently, courses of 1 or 2 injections should be administered at the same intervals prior to each further exposure to stress.

Preferably the subject (e.g. the race horse) which suffers from SIPH (e.g. EIPH) may be treated with two doses of whole cells of the bacterium according to the present invention with the second dose being administered within 1 month of the fresh application of stress (e.g. the next horse race). If, however, the second does is administered more than 1 month before the fresh application of stress (e.g. the next horse race) then a further dose may be administered within the month, preferably within 1-3 weeks prior to a fresh application of stress (e.g. before the horse race).

Preferably the subject is young and/or is young to SIPH such that irreversible tissue remodelling is unlikely to have occurred. The term “young to SIPH” as used herein means that the subject has only displayed symptoms of SIPH, such as EIPH, for a short period of time, (for example less than about 5 years, preferably less than about three years, more preferably less than about two years) and/or on a series of separate occasions close together so that the next occasion occurs before the healing response to the previous attack has been completed. The term “young” as used herein when applied to animals (e.g. horses) refers to a mammal (e.g. a horse) less than about seven years old. Preferably less than about five years old. Preferably less than about four years old. Preferably less than about three years old. More preferably less than about 2 years old.

Advantageously, the compositions of the present invention have an enhanced effect on a subject that has not undergone irreversible tissue remodelling.

An alternative strategy, in view of the commonness of the EIPH, might be to administer a course prior to the first race of a young animal. Such an animal, given suitable boosting injections, might be protected from developing the condition.

The term “protected” as used herein means that the subject is less susceptible to the disease/disorder as compared with a subject not treated or administered with the compositions according to the present invention and/or that the subject is more able to counter or overcome the disease/disorder as compared with a subject not treated or administered with the compositions according to the present invention.

In an alternative embodiment, the composition may be administered to the subject following the onset of the symptoms, for example whilst they are manifest after a race. In this embodiment preferably the subject, for instance the horse may have previously received an immunising course of the composition.

The term “whole cell”, as used herein, means a bacterium which is intact, or substantially intact. In particular, the term “intact” as used herein means a bacterium which is comprised of all of the components present in a whole cell, particularly a whole, viable cell, and/or a bacterium which has not been specifically treated to remove one or more components from it. By the term “substantially intact” as used herein it is meant that although the isolation and/or purification process used in obtaining the bacterium may result in, for example, a slight modification to the cell and/or in the removal of one or more of the components of the cell, the degree to which such a modification and/or removal occurs is insignificant. In particular, a substantially intact cell according to the present invention has not been specifically treated to remove one or more components from it.

For the avoidance of doubt, when it is the case that the bacterium is killed prior to use, for example by heat-treatment, such heat treatment may inactivate or destroy constituents of the bacterium. Such a killed, for example heat treated, bacterium may still be considered as a substantially intact whole cell in accordance with the present invention.

WO2004/022093 and WO2005/049056 (both of which references are incorporated herein by reference) disclose a composition (e.g. a pharmaceutical composition) comprising a whole cell of a bacterium from the genera Rhodococcus, Gordonia, Nocardia, Dietzia, Tsukamurella and Nocardioides. However, neither of these documents teaches or suggests the use of such an immune modulator composition or pharmaceutical composition for the treatment and/or prevention of the clinical syndrome of stress-induce pulmonary haemorrhaging (particularly exercise-induced pulmonary haemorrhaging).

Mycobacterium vaccae, or part thereof (as taught in WO2002/032455) has been used in a vaccine against mycobacterial disease (e.g. tuberculosis). However the use of M. vaccae in the prevention or treatment of SIPH is neither taught nor suggested.

Suitably, the composition or pharmaceutical composition used herein may comprise a pharmaceutically acceptable carrier, diluent or excipient.

Suitably, the composition and/or pharmaceutical composition may comprise more than one whole cell, and more preferably comprises a plurality of whole cells.

In one aspect, the composition and/or a pharmaceutical composition comprising a whole cell of a bacterium from a genus of aerobic organisms in the order Actinomycetales may further comprise at least one, or at least one further, antigen or antigenic determinant.

The composition used in accordance with the present invention may be a vaccine. The vaccine may be a prophylactic vaccine or a therapeutic vaccine.

Suitably, the composition for use in accordance with the present invention may comprise two or more, or three or more, bacteria from a genus of aerobic organisms in the order Actionomycetales.

Preferably, the bacteria for use in accordance with the present invention are species which can be grown on a medium, which is a low, preferably non-antigenic medium. By way of example only, a suitable non-antigenic medium is Sauton's medium.

The term “subject”, as used herein, means an animal. Preferably, the subject is a mammal, bird, fish or crustacean including for example livestock and humans. Preferably, the subject referred to is a racing animal. It is intended that the present invention could be used in the treatment of related syndromes in subjects other than racing animals. Hence, the present invention is effective for the treatment and/or prevention and/or reduction of stress-induced pulmonary haemorrhaging, particularly exercise-induced pulmonary haemorrhaging, in horses, greyhounds, camels and humans for example. The present invention may also be effective for the treatment and/or prevention and/or reduction of stress-induced pulmonary haemorrhaging in fish or crustaceans, for example in koi carp. However, should this syndrome or a similar syndrome be identified in another subject, such as a different animal, it is envisaged that the composition and/or pharmaceutical composition taught herein would be effective to treat and/or prevent such a syndrome in other subjects, such as in other animals. In one embodiment preferably the subject is a horse, more preferably a race horse.

Preferably, the bacterium according to the present invention is killed prior to use. Preferably, the bacterium according to the present invention is killed by heat-treatment thereof, for example, heat-treatment in an autoclave at 121° C. for 15 minutes.

Other suitable treatments for killing the bacterium may include ultraviolet or ionising radiation or treatment with chemicals such as phenol, alcohol or formalin. Suitably the ionising radiation may be carried out by exposure to 2.5 Mrads from a Co₆₀ source.

Preferably, the bacterium according to the present invention is purified and/or isolated.

Preferably, the bacterium according to the present invention is suspended in water or buffered saline, suitably borate buffered at pH 8.

### Vaccines

The compositions of the present invention may be used as a SIPH vaccine.

The preparation of vaccines which contain one or more substances as an active ingredient(s) is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the active ingredient(s) encapsulated in liposomes. The active ingredients are often mixed with excipients which are pharmaceutically acceptable and compatible with the active ingredient. Suitable excipients are, for example, water, saline, dextrose, glycerol, ethanol, or the like and combinations thereof. Alternatively, the vaccine may be prepared, for example, to be orally ingested and/or capable of inhalation.

In addition, if desired, the vaccine may contain minor amounts of auxiliary substances such as wetting or emulsifying agents and pH buffering agents.

### Administration

Typically, a physician will determine the actual dosage of the composition or pharmaceutical composition which will be most suitable for an individual subject and it will vary with the age, weight and response of the particular subject. The dosages below are exemplary of the average case. There can, of course, be individual instances where higher or lower dosage ranges are merited.

Preferably, the actual dosage that is used results in minimal toxicity to the subject.

The compositions of the present invention may be administered by direct injection. The composition may be formulated for parenteral, mucosal, intramuscular, intravenous, subcutaneous, intraocular, intradermal or transdermal administration.

Suitably, the composition according to the present invention may be administered at a dose of 10³-10¹¹ organisms, preferably 10⁴-10¹⁰ organisms, more preferably 10⁶-10-5×10⁹ organisms, and even more preferably 10⁷-2×10⁹ organisms. Typically, the composition according to the present invention may be administered at a dose of 10⁸-2×10⁹ bacteria for human and animal use.

If the compositions of the present invention are to be administrated as immune enhancers, then 10³-10¹¹ organisms per dose, preferably 10⁴-10¹⁰ organisms per dose, more preferably 10⁶-5×10⁹ organisms per dose, and even more preferably 10⁷-2×10⁹ organisms per dose, and even more preferably, 10⁸-2×10⁹ bacteria per dose for human and animal use may be administered at determined intervals.

As will be readily appreciated by a skilled person the dosage administered will be dependent upon the organism to which the dose is being administered.

The term “administering” as used herein refers to administration of bacteria of the present invention for the purposes of providing a medicament. Preferably, “administering” relates to administration for the purpose of preventing, treating and/or controlling SIPH. In other words, in one embodiment the term “administering” means that the bacteria is given (preferably as a medicament) to the subject, i.e. does not encompass the situation where the subject may comprise or acquire the bacteria naturally.

The term “administered” includes delivery by delivery mechanisms including injection, lipid mediated transfection, liposomes, immunoliposomes, lipofectin, cationic facial amphiphiles (CFAs) and combinations thereof, or even viral delivery. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular, intradermal or subcutaneous route.

The term “co-administered” means that the site and time of administration of each of the compositions, adjuvants(s), antigen(s) and/or antigenic determinant(s) of the present invention are such that the necessary modulation of the immune system is achieved. Thus, whilst the composition and either an antigen(s) and/or adjuvant(s) may be administered at the same moment in time and at the same site, there may be advantages in administering the composition and/or antigen(s) and/or antigenic determinant(s) at a different time and to a different site from the adjuvant(s). The composition and/or antigen(s) and/or antigenic determinant(s) and adjuvant(s) may even be delivered in the same delivery vehicle—and the antigen(s) and/or antigenic determinant(s) and adjuvant(s) may be coupled and/or uncoupled and/or genetically coupled and/or uncoupled. By way of example only, the composition according to the present invention may be administered before, at the same time or post administration of one or more antigens or further antigens.

The composition may be administered to the host subject as a single dose or in multiple doses.

The composition and/or pharmaceutical composition for use in accordance with the invention may be administered by a number of different routes such as injection (which includes parenteral, subcutaneous, intradermal and intramuscular injection) intranasal, mucosal, oral, intra-vaginal, urethral or ocular administration.

Preferably, in the present invention, administration is by injection. More preferably the injection is intradermal.

Preferably, in the present invention, administration is by an orally acceptable composition.

For vaccination the composition can be provided in 0.1 to 0.2 ml of aqueous solution, preferably buffered physiological saline, and administered parenterally, for example by intradermal inoculation. The vaccine according to the invention is preferably injected intradermally. Slight swelling and redness, sometimes also itching may be found at the injection site. The mode of administration, the dose and the number of administrations can be optimised by those skilled in the art in a known manner.

### Antigens

As used herein, an “antigen” means an entity which, when introduced into an immunocompetent host, modifies the production of a specific antibody or antibodies that can combine with the entity, and/or modifies the relevant T-helper cell response, such as Th2 and/or Th1. The antigen may be a pure substance, a mixture of substances or soluble or particulate material (including cells or cell fragments or cell sonicate). In this sense, the term includes any suitable antigenic determinant, cross reacting antigen, alloantigen, xenoantigen, tolerogen, allergen, hapten, and immunogen, or parts thereof, as well as any combination thereof, and these terms are used interchangeably throughout the text.

The term “antigenic determinant or epitope” as used herein refers to a site on an antigen which is recognised by an antibody or T-cell receptor, or is responsible for evoking the T-helper cell response. Preferably it is a short peptide derived from or as part of a protein antigen. However the term is also intended to include glycopeptides and carbohydrate epitopes. The term also includes modified sequences of amino acids or carbohydrates which stimulate responses which recognise the whole organism.

A “preventative” or “prophylactic” vaccine is a vaccine which is administered to naive individuals to prevent development of a condition, such as by stimulating protective immunity.

A “therapeutic” vaccine is a vaccine which is administered to individuals with an existing condition to reduce or minimise the condition or to abrogate the immunopathological consequences of the condition.

### Adjuvants

The term ‘adjuvant’ as used herein means an entity capable of augmenting or participating in the influencing of an immune response. An adjuvant is any substance or mixture of substances that assists, increases, downregulates, modifies or diversifies the immune response to an antigen.

The composition and/or pharmaceutical composition according to the present invention may comprise one or more adjuvants which enhance the effectiveness of the composition and/or pharmaceutical compositions. Examples of additional adjuvants which, may be effective include but are not limited to: aluminium hydroxide, aluminium phosphate, aluminium potassium sulphate (alum), beryllium sulphate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, Mycobacterium vaccae, polyribonucleotides, sodium alginate, lanolin, lysolecithin, vitamin A, interleukins such as interleukin 2 and interleukin-12, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Only aluminium hydroxide is approved for human use. Some of the other adjuvants, such as M. vaccae for example, have been approved for clinical trials.

Suitably, the adjuvant may be a whole cell of a bacterium from a genus of aerobic organisms in the order Actinomycetales.

In the art, it is known that DNA vaccines, which are essentially DNA sequences attached to gold particles and which are fired into the skin by a helium gun, are efficient vaccine delivery systems. Unlike conventional vaccines, these DNA vaccines do not require a traditional adjuvant component. In accordance with a further aspect of the present invention, the composition as defined herein may suitably be used in conjunction with such DNA vaccines to augment or participate in the influencing of an immune response.

### Pharmaceutical Compositions

The present invention also provides a pharmaceutical composition comprising a therapeutically effective amount of a whole cell of a bacterium from a genus of aerobic organisms in the order of Actinomycetales and optionally a pharmaceutically acceptable carrier, diluent or excipients (including combinations thereof).

The pharmaceutical composition may comprise two components—a first component comprising an antigen and a second component comprising an adjuvant thereof. The first and second component may be delivered sequentially, simultaneously or together, and even by different administration routes.

Suitably, the antigen may even be engendered within the host tissues as part of a disease process. Thus, antigen may originate from a bacterial, host or parasitic invasion, or may be a substance released from the tissues such as a stress protein, equivalent to the heat-shock proteins of bacteria or a tumour antigen.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice.

The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular, intradermal or subcutaneous route. Alternatively, the formulation may be designed to be delivered by both routes.

Preferably in the present invention the formulation is of injectable form. More preferably the formulation is intradermally injected.

Preferably in the present invention the formulation is an orally acceptable composition.

Where the agent is to be delivered mucosally through the gastrointestinal mucosa, it should be able to remain stable during transit through the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly, intradermally or subcutaneously. For parenteral administration, the compositions may be best used in the form of a sterile aqueous solution which may contain other substances, for example enough salts of monosaccharides to make the solution isotonic with blood. For buccal or sublingual administration the compositions may be administered in the form of tablets or lozenges which can be formulated in a conventional manner, or the compositions may be administered by incorporation into the food and/or feed of the subject.

### Pharmaceutical Combinations

The agent of the present invention may be administered with one or more other pharmaceutically active substances. By way of example, the present invention covers the simultaneous, or sequential treatments with a composition and/or pharmaceutical composition according to the present invention, and one or more steroids, analgesics, antivirals, interleukins such as IL-2, or other pharmaceutically active substance(s).

It will be understood that these regimes include the administration of the substances sequentially, simultaneously or together.

### Immune Enhancer

The term “immune enhancer” as used herein means one or more bacteria either isolated or in culture which when administered to a subject benefit the health of that subject. Preferably, this benefit is achieved by the modification of the cellular immune response of the subject.

In accordance with the present invention, immune enhancers may be used for the treatment and/or prevention of stress-induced pulmonary haemorrhaging, particularly exercise-induced pulmonary haemorrhaging.

The immune enhancers may be administered by consumption in specially designed food or in animal feeds, for example animal feeds supplemented with the bacteria of the present invention.

The immune enhancers may also be administered by other routes—such as direct injection.

Preferably, the bacteria are killed so as to avoid the difficulties of maintaining live products and/or to expose immunologically active substances often hidden in live bacteria.

Identifying a Bacterium that can be Used to Treat Stress-Induced Pulmonary Haemorraging

In another aspect, the present invention relates to a method for identifying one or more whole cells of bacteria from a genus of aerobic organisms in the order Actinomycetales that can treat and/or prevent stress-induced pulmonary haemorrhaging (SIPEH), particularly exercise-induced pulmonary haemorrhaging (EIPH), comprising the steps of: (a) administering a first group of test animals with an immunostimulant; (b) administering a second group of test animals with an immunostimulant mixed with a bacterium from a genera of aerobic organisms in the order Actinomycetales; (c) measuring the number or occurrences of and/or severity of SIPH (preferably EIPH) in each of the test animals; and (d) comparing the results in each of the groups of test animals, wherein, a lower occurrence of and/or severity of SIPH (preferably EIPH) from the immunostimulant mixed with a bacterium in comparison to the innunostimulant alone is indicative of a bacterium suitable for use in accordance with the present invention.

As used herein, the term “test animal” refers to any animal that elicits a cellular immune response to the immunostimulant. Preferably, the test animal(s) is a mammal.

Preferably, the bacterium modifies the T helper cell response. Suitably, the bacterium may modify the T helper cell response by increasing the Th1 response and down-regulating the Th2 response.

Preferably, the immunostimulant will induce/enhance a known Th1 and Th2 response. For example, with the immunostimulant BCG the reaction to Tuberculin is usually largest at 24 h when it is an indicator of the Th1 response; the reaction at 48 h is usually less and includes a Th2 contribution. It is known that BCG predominantly stimulates a Th1 response in a naïve animal.

By use of such immunostimulants it may be possible to determine the Th1/Th2 response of a test bacterium and, thus, it may be possible to identify one or more bacteria which have a desired Th1/Th2 response to treat and/or prevent a particular disease and/or disorder.

Preferably, the cellular immune response is measured using the tuberculin skin test. In mice, the tuberculin skin test is preferably carried out on the foot pad. In a predominant Th1 reaction the positive foot pad immune response is maximal at 24 hours and diminishes at 48 hours. However, as the Th2 reactivity increases then the 48 hour positive foot pad immune response increases and can even exceed the foot pad immune response at 24 hour.

Vaccination with an immunostimulant—such as BCG—induces a response to skin-testing with tuberculin (a soluble preparation of Tubercle bacilli), when tested later. The local reaction is measured at various intervals, for example, 24 hours, 48 hours and 72 hours after injection of tuberculin. Briefly, an immunostimulant (e.g. BCG) is used that induces a positive immune response to tuberculin. In the test animal, the tuberculin skin test is preferably carried out on the foot pad. In a predominant Th1 reaction the positive foot pad immune response is usually maximal at 24 hours and diminishes at 48 hours. However, as the Th2 reactivity increases then the 48 hour positive foot pad immune response increases and can even exceed the foot pad immune response at 24 hour. Thus, the assay can be used to assess whether or not the introduction of an immune modulator composition according to the present invention modulates the cellular immune response.

Preferably, the immunostimulant is BCG.

The invention will now be further described by way of Examples, which are meant to serve to assist one of ordinary skill in the art in carrying out the invention and are not intended in any way to limit the scope of the invention.

### EXAMPLES

### Example 1

Exercise-induced pulmonary haemorrhage (EIPH) is a common condition afflicting up to 85% of equine athletes. It varies from minor bleeding detected by observing red blood cells in broncho-alveolar fluid obtained by fibroscopy, to frank blood appearing in mucus froth around the nostrils at the end of a race. Horses prone to the condition suffer these symptoms after every race, which slows down the horse in the final furlongs.

The precise cause of the condition is uncertain. Without wishing to be bound by theory, we believe that pulmonary hypertension may be responsible or partly responsible for this condition. For example, it is hypothesised that under increased pressure the pulmonary arterioles and capillaries rupture, often also damaging the alveolar architecture and progressively reducing the pulmonary reserve with accompanying pulmonary fibrosis. At an arterial level repeated episodes of pulmonary hypertension may result in intimal damage and myointimal hyperplasia, accentuating the condition.

Without wishing to be bound by theory, the inventors now believe that allergic responses may also play a part and eosinophils often infiltrate between the alveoli of horses showing EIPH, suggesting that exercise-induced asthma may also contribute to the condition.

The inventors now believe that when inflammation of the arterial wall due to raised puhnonary arterial pressure coincides with alveolar membranes damaged by allergic processes, excessive bleeding may result.

There is no known cure for the condition and although furosamide reduces the problem, traces detected before a race disqualifies the horse.

As the inventors now believe that SIPH (particularly EIPH) may be caused by a combination of arterial damage, and pulmonary damage due to an allergic response for example, the inventors investigated whether SIPH (particularly EIPH) responds to immunomodulation in which Th1 mechanisms are enhanced and Th2 mechanisms are down-regulated.

A horse suffering from EIPH after every race was treated with 2 doses of 1-2 mg whole cells of Tsukamurella inchonensis per dose, 3 weeks apart and then raced a few weeks later. The horse showed complete alleviation of the condition.

### Example 2

### Comparative Study on Bronchoalveolar Lavages from Horses with Non Infectious Pulmonary Inflammation Under an Immunisation Protocol

### Introduction

Young horses undergoing turf sport activities experience environmental changes resulting in the establishment of a non-infectious pulmonary inflammation, among other disorders. Data from the literature indicate that during maximal intensity exercises a reduction in horse performance ensues, in many cases leading to sub-clinical stages not easily diagnosed and therefore difficult to treat efficaciously.

Given this background in the present study we decided to analyze whether treatment with an immunotherapeutic preparation was likely to modify the cell composition of inflammatory infiltrates of broncho-alveolar lavages (BAL) from such kind of horses.

### Materials and Methods

The study is carried out in six male racing horses (mean weight 460±40 Kg) aged 2.5±0.5 years on average. All horses presented a non-infectious pulmonary inflammation which was diagnosed by three consecutive cytologic examinations from BAL samples. Measurements were separated by a 20-day interval.

Horses are allocated in individual boxes, in the area where the La Plata racetrack is situated. Beds were prepared with shavings, and horse are fed with (alfalfa) lucerne hay, oat and water. Environmental quality was related to bed cleaning, ventilation and food quality, being categorized as of median quality.

BALs were performed with 300 ml sterile physiologic saline (at the body temperature), given in 5 aliquots, the first one being not employed for the study. Samples were centrifuged until the macroscopic sediment was obtained (10 min at 1500 rpm twice) to be further stained with 15-staining and then subjected to differential cell counts.

### Results

Differential cell counts in three BAL samples obtained before immunisation (interval between sample collections was 20 days—Time 0, Time 1 and Time 2), after first Tsukamurella inchonensis injection (Time 3) and after second Tsukamurella inchonensis injection (Time 4).

Horse 1

| BAL | MA | MI | L | Mast | N | Epit | H |
|---|---|---|---|---|---|---|---|
| Time 0 | 22 | 16 | 47 | 9 | 4 | 2 | 0 |
| Time 1 | 26 | 19 | 30 | 16 | 6 | 1 | 2 |
| Time 2 | 10 | 21 | 35 | 19 | 7 | 4 | 4 |
| Time 3 | 15 | 35 | 30 | 10 | 6 | 0 | 4 |
| Time 4 | 15 | 32 | 33 | 8 | 8 | 2 | 2 |

Horse 2

| BAL | MA | MI | L | Mast | N | Epit | H |
|---|---|---|---|---|---|---|---|
| Time 0 | 12 | 36 | 35 | 10 | 2 | 3 | 1 |
| Time 1 | 15 | 39 | 32 | 6 | 2 | 4 | 2 |
| Time 2 | 7 | 42 | 26 | 15 | 5 | 2 | 3 |
| Time 3 | 20 | 35 | 29 | 8 | 5 | 1 | 2 |
| Time 4 | 12 | 42 | 31 | 8 | 5 | 0 | 2 |

Horse 3

| BAL | MA | MI | L | Mast | N | Epi | H |
|---|---|---|---|---|---|---|---|
| Time 0 | 22 | 15 | 39 | 12 | 8 | 3 | 1 |
| Time 1 | 13 | 38 | 32 | 8 | 6 | 3 | 0 |
| Time 2 | 16 | 15 | 36 | 14 | 12 | 5 | 2 |
| Time 3 | 18 | 47 | 20 | 6 | 6 | 2 | 1 |
| Time 4 | 2 | 58 | 31 | 2 | 5 | 2 | 0 |

Horse 4

| BAL | MA | MI | L | Mast | N | Epi | H |
|---|---|---|---|---|---|---|---|
| Time 0 | 36 | 22 | 24 | 8 | 6 | 2 | 2 |
| Time 1 | 39 | 26 | 10 | 18 | 4 | 3 | 0 |
| Time 2 | 36 | 35 | 12 | 12 | 3 | 2 | 0 |
| Time 3 | 10 | 48 | 39 | 1 | 2 | 0 | 0 |
| Time 4 | 15 | 41 | 35 | 1 | 5 | 2 | 1 |

Horse 5

| BAL | MA | MI | L | Mast | N | Epit | H |
|---|---|---|---|---|---|---|---|
| Time 0 | 22 | 28 | 40 | 9 | 1 | 0 | 0 |
| Time 1 | 18 | 23 | 37 | 15 | 5 | 1 | 1 |
| Time 2 | 14 | 23 | 41 | 13 | 6 | 3 | 0 |
| Time 3 | 8 | 56 | 35 | 0 | 1 | 0 | 0 |
| Time 4 | 10 | 40 | 42 | 2 | 4 | 2 | 1 |

Horse 6

| BAL | MA | MI | L | Mast | N | Epit | H |
|---|---|---|---|---|---|---|---|
| Time 0 | 30 | 22 | 38 | 9 | 1 | 0 | 0 |
| Time 1 | 40 | 20 | 32 | 6 | 0 | 1 | 1 |
| Time 2 | 26 | 27 | 25 | 15 | 4 | 3 | 0 |
| Time 3 | 5 | 55 | 36 | 1 | 3 | 0 | 0 |
| Time 4 | 12 | 48 | 36 | 1 | 3 | 0 | 0 |

References and normal values:
MA: active macrophages <5%
MI: inactive macrophages 40-60%
L: Lymphocytes 20-40%
Mast: Mast cells <2%
N: PMN <5%
Epit: epithelial cells <0.5%
H: Hemosiderophages <1%

Upon obtaining the third sample (Time 2), horses were injected with Tsukamurella inchonensis and sampled 20 days later (Time 3).

A second with Tsukamurella inchonensis injection was given three weeks after the 1st one.

An additional sample (Time 5) will be taken 20 days later.

After an initial injection with Tsukamurella inchonensis 3 of the 6 horses showed an improvement. After the second injection such 3 horses continued to improve and an additional horse also improved. Improvement was based on the reduction of inflammatory cells dealing with allergic reactions, such as mast cells.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. Use of a composition comprising whole cells of bacteria from a genus of aerobic organisms in the order of Actinomycetales in the manufacture of a medicament for the treatment or prevention of stress-induced pulmonary haemorrhage.

2. Use according to claim 1 wherein said stress-induced pulmonary haemorrhage is exercise-induced pulmonary haemorrhage.

3. Use according to claim 1 wherein said bacteria may be selected from any mycolic-acid containing bacteria.

4. Use according to claim 3 wherein said bacteria are selected from any one or more of the following genera: Tsukamurella, Rhodococcus, Gordonia, Nocardia, Dietzia, and Mycobacterium.

5. Use according to claim 4 wherein said bacteria are from one or more of the following species: Tsukamurella inchonensis, Tsukamurella paurometabola. Gordonia bronchialis, G. amarae, G. sputi, G. terrae, Nocardia asteroides, N. brasiliensis, Dietzia maris, Rhodococcus ruber, R. rhodnii, R. coprophilus, R. opacus, R. erythopolis, Mycobacterium vaccae and M. obuense.

6. Use according to claim 4 wherein said bacteria are from Rhodococcus.

7. Use according to claim 6 wherein said bacteria are from Rhodococcus coprophilus.

8. Use according to claim 4 wherein said bacteria are from Tsukamurella.

9. Use according to claim 8 wherein said bacteria are from Tsukamurella inchonensis.

10. Use according to claim 4 wherein said bacteria are from Mycobacterium.

11. Use according to claim 10 wherein said bacteria are from Mycobacterium vaccae and/or Mycobacterium obuense.

12. Use according to claim 1 wherein said bacteria are killed.

13. A method for treating or preventing stress-induced pulmonary haemorrhage in a subject comprising administering an effective amount of a composition comprising whole cells of bacteria from a genus of aerobic organisms in the order of Actinomycetales, to said subject.

14. The method according to claim 13 wherein said stress-induced pulmonary haemorrhage is exercise-induced pulmonary haemorrhage.

15. The method according to claim 13 or wherein said bacteria may be selected from any mycolic acid containing bacteria.

16. The method according to claim 15 wherein said bacterium is one or more of the following genera: Tsukamurella, Rhodococcus, Gordonia, Nocardia, Dietzia and Mycobacterium.

17. The method according to claim 16 wherein said bacterium is one or more of the following species: Tsukamurella inchonensis, Tsukamurella paurometabola, Gordonia bronchialis, G. amarae, G. sputi, G. terrae, Nocardia asteroides, N. brasiliensis, Tsukamurella paurometabola, Rhodococcus ruber, Rhodococcus rhodnii, R. coprophilus, R. opacus, R. erythopolis, Dietzia maris, Mycobacterium vaccae and M. obuense.

18. The method according to claim 16 wherein said bacterium is from Rhodococcus.

19. The method according to claim 18 wherein said bacterium is from Rhodococcus coprophilus.

20. The method according to claim 16 wherein said bacterium is from Tsukamurella.

21. The method according to claim 20 wherein said bacterium is from Tsukamurella inchonensis.

22. The method according to claim 16 wherein said bacterium is from Mycobacterium.

23. The method according to claim 22 wherein said bacterium is from Mycobacterium vaccae and/or Mycobacterium obuense.

24. The method according to claim 13 wherein said bacteria are killed.

25. A pharmaceutical pack for use in the treatment of stress-induced pulmonary haemorrhage wherein at least one compartment comprises whole cells of bacteria from a genus of aerobic organisms in the order Actinomycetales.

26. A pharmaceutical pack according to claim 25 wherein said pack comprises a label stating that it is suitable for use in the prevention or treatment of stress-induced pulmonary haemorrhage.

27.-29. (canceled)
